(19) United States
(12) Patent Application Publication
(10) Pub. No.: US 2013/0281944 A1
(43) Pub. Date: Oct. 24, 2013

(54) TRANSDERMAL THERAPEUTIC SYSTEM (TTS) COMPRISING ROTIGOTINE

(71) Applicants: Christian Drescher, Ulm (DE); Christian Janssen, Illerkirchberg (DE); Ulrich Becker, Langenfield (DE); Armin Breitenbach, Leverkusen (DE)

(72) Inventors: Christian Drescher, Ulm (DE); Christian Janssen, Illerkirchberg (DE); Ulrich Becker, Langenfield (DE); Armin Breitenbach, Leverkusen (DE)

(73) Assignee: Ratiopharm GMBH, ULM (DE)

(21) Appl. No.: 13/990,115

(22) PCT Filed: Nov. 29, 2011
(86) PCT No.: PCT/EP11/71321

§ 371 Date: Jul. 2, 2013

Related U.S. Application Data
(60) Provisional application No. 61/419,070, filed on Dec. 2, 2010.

(30) Foreign Application Priority Data
Nov. 29, 2010 (EP) 10015079.6

(51) Int. Cl. A61M 35/00

(52) U.S. Cl. CPC A61M 35/00
USPC 604/307

(57) ABSTRACT

The present invention relates to a patch containing the active ingredient rotigotine for the transdermal delivery of the active pharmaceutical agent rotigotine, comprising a backing layer (1), a matrix layer (2) containing the active ingredient, and a removable protective sheet (4) which is intended for removal before the patch is applied to the skin, characterized in that disposed with areal coverage between the matrix layer (2) and the removable protective sheet (4) is an additional interlayer (3).

### FIELD OF THE INVENTION

The present invention relates to a patch containing the active ingredient rotigotine for the transdermal delivery of the active pharmaceutical agent rotigotine, comprising a backing layer (1), a matrix layer (2) containing the active ingredient, and a removable protective sheet (4) which is intended for removal before the patch is applied to the skin, characterized in that disposed with areal coverage between the matrix layer (2) and the removable protective sheet (4) is an additional interlayer (3).

### DESCRIPTION

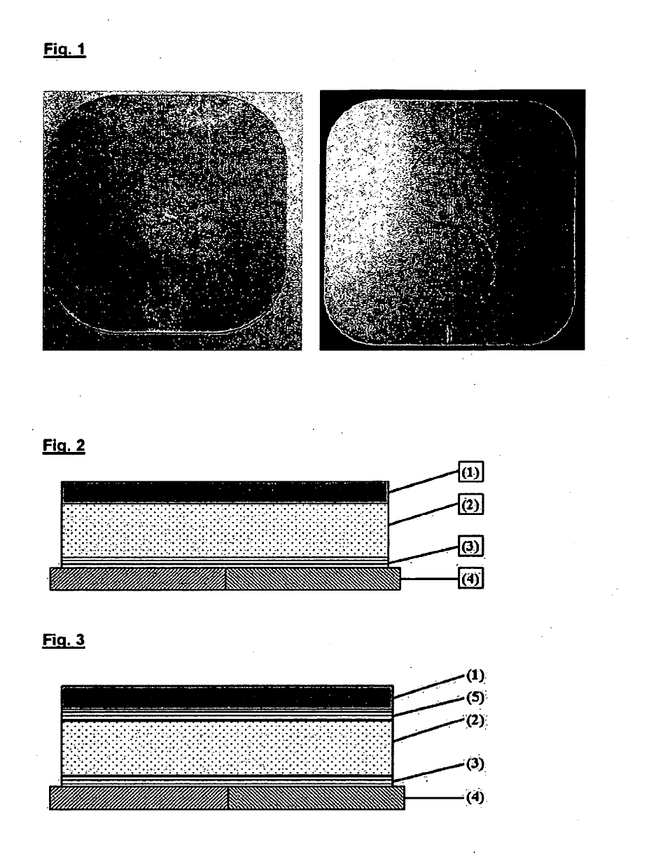

Rotigotine [(−)-5,6,7,8-tetrahydro-6-[propyl [2-(2-thienyl)ethyl]amino]-1-naphtol] is an active pharmaceutical ingredient which is used, inter alia, in the treatment of Parkinson's disease. The only currently available pharmaceutical product containing rotigotine is a transdermal therapeutic system (TTS). Neupro® by UCB contains the active ingredient embedded in a polymer matrix in an amount exceeding the saturation concentration, thus rendering it a so-called “supersaturated system”. Supersaturated active ingredient/polymer systems are known to be metastable, i.e. the active ingredient may crystallize under unfavorable conditions: a problem that has already led to a product recall of Neupro® because the active ingredient had crystallized out of the patches (see FIG. 1). Crystallization of the active ingredient may generate a plurality of problems: for instance, a reduced amount of active ingredient is available for skin absorption, so that the intended delivery rate can no longer be ensured. The crystallization of the active ingredient may also negatively influence the adhesive properties (adhesion and cohesion, respectively) of the adhesive matrix. To this day, a cold chain is required for the transport and storage of Neupro® and it is further recommended to store Neupro® in a refrigerator.

There have been suggestions in the prior art, e.g. in WO 99/49852 or WO 2004/058247, to produce rotigotine patches using so-called matrix systems in which the active ingredient is incorporated in a matrix made of a pressure-sensitive polymer adhesive. However, the selection of the polymer adhesives to be used is always associated with a compromise as the respective adhesive is required to have both sufficient solubility with respect to the active ingredient and adequate adhesive properties. The development of a suitable TTS is rendered even more complicated by the fact that active ingredients in higher concentrations (Neupro®, for instance, contains rotigotine base in a proportion of about 9% by weight) can significantly influence the adhesive properties of polymer matrices, which may lead to problems with respect to adhesion and cohesion as well as to the occurrence of a phenomenon called “cold flow”. Furthermore, self-adhesive polymers often contain free functional groups, such as hydroxyl (OH) or carboxyl (COOH) groups, which interact with the active ingredient and can thus negatively influence the crystallization and release behavior thereof.

Based on the prior art, it is therefore an object of the present invention to provide a rotigotine TTS that independently facilitates the optimization of both adhesive properties and release behavior.

This object has been achieved by providing a transdermal therapeutic system (TTS) containing the active ingredient rotigotine and having a backing layer (1) which is impermeable to the active ingredient, a matrix layer (2) containing the active ingredient, and a removable protective sheet (4) which is intended for removal before the patch is applied to the skin, characterized in that disposed with areal coverage between the matrix layer (2) and the removable protective sheet (4) is an additional interlayer (3).

The matrix layer (2) determines the release properties of the active ingredient while the adhesive properties of the TTS are determined by the selection of a suitable interlayer (3). The composition of the TTS according to the present invention thus facilitates the independent modulation of both the release properties of the active ingredient and the adhesive properties of the patch.

As such a system also allows for the use of non-self-adhesive polymers for forming the matrix layer (2), the possibilities of selecting the polymers that can be used for the reservoir are significantly increased. According to the present invention, non-self-adhesive polymers are polymers whose adhesive strength is not sufficient to ensure that a TTS according to the present invention will adhere to the human skin over the entire administration period without any further auxiliary means. Examples for non-self-adhesive polymers are polymers having longer aliphatic chains and/or polymers lacking functional groups.

Functional groups, such as e.g. carboxyl groups, which are usually responsible for the adhesive properties of a polymer, can potentially interact with basic active ingredients, such as rotigotine, in an undesired manner, e.g. by the formation of salts. In contrast thereto, polymers lacking these functional groups can have preferred solubility and release properties with respect to the active ingredient.

A preferred embodiment of the present invention thus relates to a TTS having a backing layer (1) which is impermeable to the active ingredient, a matrix layer (2) containing the active ingredient, a removable protective sheet (4) which is intended for removal before the patch is applied to the skin, and an interlayer (3) which is disposed with areal coverage between the matrix layer (2) and the removable protective sheet (4), characterized in that the matrix layer (2) consists of a non-self-adhesive polymer.

It is a further advantage of the present invention that for forming the interlayer (3) also polymers are used that may not readily be considered as a suitable material for the reservoir (2) due to their poor solubility.

FIG. 2 schematically shows the composition of a TTS having a self-adhesive matrix layer, containing:
a backing layer (1) which is impermeable to the active ingredient;
a self-adhesive matrix layer (2) containing rotigotine;
an interlayer (3) which is disposed with areal coverage between the matrix layer (2) and the removable protective sheet (4); and
at least one removable protective sheet (4) which is intended for removal before the patch is applied to the skin.

FIG. 3 schematically shows the composition of a TTS having a non-adhesive matrix layer, containing:
a backing layer (1) which is impermeable to the active ingredient;
a non-adhesive matrix layer (2) containing rotigotine;
a first interlayer (3) which is disposed with areal coverage between the matrix layer (2) and the removable protective sheet (4);
at least one removable protective sheet (4) which is intended for removal before the patch is applied to the skin; and
a second interlayer (5) which is disposed with areal coverage between the matrix layer (2) and the backing layer (1).

Furthermore, the comprehensive shielding of the matrix layer (2) by the interlayer (3) has the additional advantage of avoiding direct contact between the reservoir and the cut edges of the removable protective sheet (4) which is intended for removal before the patch is applied to the skin—i.e. the obvious reason for the crystallization in case of the commercial product Neupro® (see FIG. 1).

The backing layer (1) of the patch according to the present invention, which is impermeable to the active ingredient, is impermeable and inert with respect to both the active ingredient and the compounds contained in the layer containing the active ingredient, i.e. there is no migration into the backing layer. The backing layer which is impermeable to the active ingredient may be permeable to air and water, but is preferably occlusive. On the one hand, the backing layer has to provide a certain stiffness in order to sufficiently stabilize the matrix layer (2) containing the active ingredient and thus render the TTS easy to handle, and on the other hand it has to provide a certain sufficient flexibility, so that even larger TTS patches are still capable of adapting their shape to the skin, thus ensuring sufficient wearing comfort.

The backing layer (1) which is impermeable to the active ingredient consists of polymers, such as polyesters (e.g. PET), polyolefines (e.g. PE), polycarbonates, polyethylene oxides, polyurethanes, polystyrenes, polyamides, polyvinyl acetates and polyvinyl chlorides. An aluminum vapor-coated PE/PET backing layer is preferred (e.g. Scotchpak 1109, 3M).

Film-forming polymers, such as e.g. polyacrylates, ethylene/ethyl acrylate copolymers, polyisobutylenes (PIB), and polysiloxanes are used as materials for forming the matrix layer (2) containing rotigotine. Preferred are acrylate copolymers, the kind such as, e.g. Eudragit®.

In one embodiment, rotigotine is preferably used in the form of the free rotigotine base.

In addition to the pharmaceutically active ingredient rotigotine, the matrix layer (2) containing the active ingredient may also contain excipients, such as anti-oxidants, diluents, permeation-enhancing agents, solubilizers, cross-linking agents, emulsifiers, preservatives and/or thickening agents.

The active ingredient may be present in the matrix layer (2) in a dissolved and/or dispersed state. Preferably, the active ingredient is present in a completely dissolved state.

The solubility of the matrix layer (2) with respect to the active ingredient rotigotine is in a range between 4 and 30% by weight, preferably between 6 and 20% by weight, even more preferably between 8 and 15% by weight.

The content of the active ingredient rotigotine in the matrix layer (2) is in a range between 4 and 30% by weight, preferably between 6 and 20% by weight, even more preferably between 8 and 15% by weight in relation to the total weight of the matrix layer.

Polymers derived from silicones, polyacrylates, polyvinyl ethers, polyisobutylenes (PIB), styrene-isoprene or butadiene-styrene copolymers, polyolefines, polyvinyl acetates, polyamides and/or ethylene-vinyl acetate copolymers are suitable materials for forming the interlayer (3).

The interlayer (3) is preferably self-adhesive and preferably consists of a pressure-sensitive polymer adhesive, preferably of an amine-resistant silicone adhesive, such as e.g. BIO-PSA Q7-4301 or BIO-PSA Q7-4201 (Dow Corning).

In addition, the interlayer (3) may optionally contain excipients, such as diluents, bulking agents, cross-linking agents, preservatives and/or solvents as well as antioxidants.

The thickness of the interlayer (3) is in a range between 4 and 25 μm, preferably between 8 and 20 μm and particularly preferably between 10 and 15 μm.

Optionally, the interlayer may simultaneously serve as a membrane for controlling the active ingredient flux, wherein the permeation of the active ingredient is reduced by not more than 20%, preferably not more than 10%.

In addition to the primary function of the interlayer, i.e. regulating the adhesive strength, its interaction with the active ingredient should be kept as low as possible. Therefore, the solubility of the interlayer with respect to the active ingredient is preferably in a range of only 5% to 15% of the solubility of the matrix layer, preferably 5% to 10%. The ratio of the thickness of the matrix layer to the thickness of the interlayer is in a range between 20:1 and 3:1, preferably between 10:1 and 5:1.

The removable protective sheet (4) which is intended for removal before the patch is applied to the skin consists of polyethylene, polypropylene, polyesters (PET), polysiloxanes, polyvinyl chloride or polyurethane or optionally of processed paper fibers and is preferably coated with silicone, fluorosilicone or fluorocarbon polymers. A fluoropolymer-coated PET liner (e.g. Scotchpak 1022, 3M) is preferred.

The second interlayer (5) may be made of substantially the same materials as the interlayer (3). Preferably, both layers consist of the same material.

The TTS preferably has a composition as depicted in FIG. 1 or FIG. 2. In addition, the TTS may have an adhesive film (overtape) by means of which the TTS is attached to the skin.

The TTS has a size of between 5 and 100 cm², preferably between 10 and 50 cm². The areal density of the TTS (without the removable protective sheet) is between 20 and 150 g/cm², preferably between 30 and 100 g/cm², particularly preferably between 40 and 70 g/cm².

In a preferred embodiment, the TTS has an adhesive strength of between 5 N/25 mm² and 100 N/25 mm², preferably between 10 N/25 mm² and 50 N/25 mm². The adhesive strength is determined according to known standard methods (e.g. ASTM D1000-99).

The patch according to the present invention is suitable for the transdermal administration of rotigotine over 12 h up to 3 days, i.e. a patch is applied over said period. An application period of 24 h to 48 h is preferred. Preferred release rates are in a range between 0.05 μg/h and 1 μg/h, preferably between 0.1 and 0.5 μg/h, over a substantial portion of the administration period.

In a preferred embodiment, the TTS provides a mean maximum plasma concentration (Cₘₐₓ) of rotigotine between 0.1 and 150 ng/mL, preferably between 0.5 and 100 ng/mL. In a further preferred embodiment, the TTS provides an AUC of between 4.0 and 30 ng*h/mL, in particular between 5.0 to 20 ng*h/mL, after repeated individual applications.

The patch according to the present invention is manufactured according to known methods. For instance, the manufacture of a rotigotine TTS according to the present invention comprises the following steps:
a. dissolving or suspending rotigotine base in a suitable solvent;
b. adding the solution/suspension to a polymer solution while stirring;
c. coating a suitable backing layer (1) with the polymer solution containing the active ingredient;
d. drying by removing the solvent, wherein a first laminate is formed by the backing layer and the matrix layer (2) containing rotigotine;
e. coating a suitable protective layer (4) with a suitable polymer solution;
f. drying by removing the solvent, wherein a second laminate is formed by the protective layer (4) and the interlayer (3) which is free of active ingredient;
g. combining both laminates so as to superimpose the matrix layer (2) and the interlayer (3) (see Figures);
h. cutting out patches of a suitable size from the combined laminate; and
i. packaging the individual TTSs in suitable containers.

In case of TTSs with a non-self-adhesive matrix layer, the backing layer (1) is first coated with a suitable polymer solution and subsequently dried before the matrix layer is applied (method step c).

One aspect of the present invention relates to a transdermal therapeutic system (TTS) containing the active ingredient rotigotine in its free base form and comprising a backing layer (1) which is impermeable to the active ingredient, a matrix layer (2) containing the active ingredient, a removable polymer-coated protective layer (4) which is intended for removal before the patch is applied to the skin and an interlayer (3) which is disposed with areal coverage between the matrix layer (2) and the removable protective sheet (4), characterized in that the matrix layer (2) consists of a pressure-sensitive polymer adhesive in which the active ingredient is present in a completely dissolved state.

One embodiment of this aspect of the present invention relates to a transdermal therapeutic system (TTS) containing the active ingredient rotigotine in its free base form, comprising a backing layer (1) which is impermeable to the active ingredient, a matrix layer (2) containing the active ingredient, a removable polymer-coated protective layer (4) which is intended for removal before the patch is applied to the skin and an interlayer (3) which is disposed with areal coverage between the matrix layer (2) and the removable protective sheet (4), characterized in that the matrix layer (2) consists of a pressure-sensitive polyacrylate adhesive and contains the completely dissolved active ingredient in an amount between 8 and 15% by weight.

In particular embodiments of this aspect of the present invention, the areal density of the TTS is between 30 g/cm² and 100 g/cm² and the ratio of the thickness of the matrix layer (2) and the thickness of the interlayer (3) is between 10:1 and 5:1.

In another embodiment of this aspect of the present invention, the transdermal therapeutic system (TTS) is characterized in that a second interlayer (5) is disposed with areal coverage between the matrix layer (2) and the backing layer (1).

As an alternative to the use of rotigotine as a free base, it may also be used in the form of an ionic liquid in the method for manufacturing a TTS according to the present invention. The term “ionic liquid”, as used herein, refers to salts (i.e. compositions comprising organic cations and organic anions) having a melting point of less than 40° C., preferably of less than 32° C. Such an ionic liquid comprises at least one rotigotine cation and at least one type of counterion, obtainable from an organic compound, or at least one rotigotine anion and at least one type of counterion, obtainable from an organic compound.

It has been found that rotigotine in the form of a cation or an anion is capable of forming ionic liquids with organic compounds, wherein said ionic liquids may easily be prepared using a plurality of pharmaceutically acceptable organic compounds. These ionic rotigotine liquids exhibit a high thermal stability and, in particular, do not give rise to problems due to crystallization that occur with the free base and other salts known in the prior art. It is a further advantage of said liquids that they do not give rise to problems with respect to the presence of polymorphic forms of the crystalline compound. Active ingredients that are present in a number of polymorphic forms are often unstable when stored in the form of pharmaceutical preparations. As alterations in the crystal form may have far-reaching consequences with respect to the physico-chemical parameters of a substance, it is advantageous to be able to bypass the solid state.

The ionic liquids are composed of at least one rotigotine cation and at least one type of anion, or alternatively of at least one rotigotine anion and at least one type of cation. It is obvious to the person skilled in the art that the ionic liquids according to the present disclosure may contain a rotigotine cation in combination with more than one type of anion (e.g. 2, 3 or more different types of anions). However, one anion is preferred. Furthermore, it is obvious to the person skilled in the art that the ionic liquids according to the present disclosure may contain a rotigotine anion in combination with more than one type of cation (e.g., 2, 3 or more different types of cations). However, one cation is preferred. The ions are preferably pharmaceutically acceptable. The use of all the anions or cations or rotigotine salts formed with said counterions (as defined in the following) in the above-defined TTSs are objects of the present invention, with the proviso that the melting point of said rotigotine salts is less than 40° C., preferably less than 32° C. (i.e. liquid rotigotine salts).

In a preferred embodiment, the stoichiometric ratio of the rotigotine ion and the respective counterion in the ionic liquid is between 1:3 and 1:1, preferably 1:2 or 1:1, and most preferably approximately 1:1.

In another embodiment, the ionic liquid comprises a rotigotine cation and at least 50% anions of an organic acid. Accordingly, compositions comprising rotigotine salts with different anions of organic and inorganic acids, such as HCl, are also encompassed by the present invention, provided that at least 50% of the anions are derived from an organic acid.

In a further embodiment, the organic compound from which the counterion is derived has a lipophilic character that is defined by a value of the distribution coefficient log P of more than 0. Preferably, said lipophilic compound has a log P value of more than 1, more preferably of more than 2, even more preferably of more than 3, and most preferably of more than 4.

In particular embodiments, the counterion as an organic compound has the following properties:

1. a distribution coefficient log P in a range between 0 and 6;

2. a molecular weight between 100 and 500;

3. a number of atoms between 20 and 70.

In a further embodiment, the present invention relates to the use of compositions comprising a rotigotine salt in the transdermal therapeutic systems (TTSs) according to the present invention, wherein at least 1%, preferably at least 2%, 5%, 10%, 20%, 30%, 40% or even 50% of the salt is present as an ionic liquid. In addition to the ionic liquid of rotigotine, such a composition may accordingly also comprise rotigotine salts, e.g. solid salts with different anions that are not present as ionic liquids. In such compositions, the content of an ionic liquid of rotigotine thus is at least 1%, preferably at least 2%, 5%, 10%, 20%, 30%, 40% or even 50% of the total content of rotigotine salt in said composition.

### Cations as Counterions

In particular embodiments of the present invention, the ionic liquid of rotigotine contains a rotigotine anion and a cationic counterion that is selected from the group of cations as defined in the following.

### Quaternary Ammonium Compounds

Specific examples of cations that may be present in the ionic liquid are compounds containing a nitrogen atom. Nitrogen atoms may be present in the form of positively charged quaternary ammonium species or may be transformed into such a form, e.g. by alkylation or protonation of the nitrogen atom. Accordingly, compounds containing a quaternary nitrogen atom (known as quaternary ammonium compounds or QACs) are typically cations. Any compound containing a quaternary nitrogen atom or a nitrogen atom that is capable of being transformed into a quaternary nitrogen atom may be a cation according to the invention for the ionic liquids disclosed.

According to the present invention, the ionic liquid comprises a rotigotine anion and at least one quaternary amine of the formula (I)

R¹R²R³R⁴N⁺ (I)

wherein R¹, R², R³, and R⁴ each independently may represent H, optionally substituted C₁-C₅ alkyl or alkenyl, optionally substituted C₆-C₁₀ cycloalkyl, optionally substituted C₆-C₁₂ aryl, optionally substituted C₇-C₁₂ aralkyl, or wherein R¹ and R² together represent an optionally substituted C₄-C₁₀ alkylene group and form a 5- to 11-membered heterocyclic ring with the nitrogen atom of formula (I), and wherein the term “optionally substituted” indicates that the respective group is either substituted with one or more residues, preferably with between 0 and 6 residues, selected from OH, SH, SR⁵, Cl, Br, F, I, NH₂, CN, NO₂, COOR⁵, CHO, COR⁵ and OR⁵, wherein R⁵ represents a C₁-C₁₀ alkyl or cycloalkyl group, or is not substituted. In a preferred embodiment, R¹ is a C₁-C₅ alkyl or alkenyl residue.

### Tetraalkyl Ammonium

In other embodiments of the present invention, the cationic counterion is a tetraalkyl ammonium cation. For instance, a tetraalkyl ammonium cation may represent a long-chain alkyl group (e.g. having a length of 10 or more carbon atoms) and three short-chain alkyl groups (e.g. having a length of less than 10 carbon atoms).

The ionic liquids may also contain an aliphatic benzyl alkyl ammonium cation. An aliphatic benzyl alkyl ammonium cation is a cation comprising an aliphatic group that is bound to the nitrogen atom of a benzyl alkylamine. The aliphatic group may be as described herein. The benzyl alkylamine group may be a benzylamine, wherein the amine is bound to an alkyl or cycloalkyl group, as described herein. One or more types of aliphatic benzyl alkyl ammonium cations may be used in the ionic liquids.

In particular embodiments of this aspect of the present invention, the aliphatic benzyl alkyl ammonium cation is represented by the following formula:

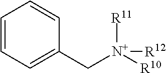

wherein R¹⁰ represents an aliphatic group, as described above, and R¹¹ and R¹² independently represent alkyl or cycloalkyl groups, as described herein. In some exemplary embodiments, one or more of the “R” substituents may be a long-chain alkyl group (i.e. with 10 or more carbon atoms). In other exemplary embodiments, one or more of the “R” substituents may be a short-chain alkyl group (i.e. with less than 10 carbon atoms). In other exemplary embodiments, one of the “R” substituents may be a long-chain alkyl group and the two other “R” substituents may be short-chain alkyl groups.

In a further embodiment, the aliphatic benzyl alkyl ammonium cation may have any of the alkyl groups as described herein bound to any benzyl alkylamine group as described herein. In specific examples, R¹⁰ in the formula of the aliphatic benzyl alkyl ammonium cation may be an aliphatic group having 10 to 40 carbon atoms, e.g. a decyl, dodecyl (lauryl), tetradecyl (myristyl), hexadecyl (palmityl or cetyl), octadecyl (stearyl), or eicosyl (arachidyl) group, and R¹¹ and R¹² may independently be a methyl, ethyl, propyl, butyl, pentyl, or hexyl group.

In other embodiments, the aliphatic benzyl alkyl ammonium cation may comprise, but is not limited to, alkyl dimethyl benzyl ammonium cations. Specific non-limiting examples of alkyl dimethyl benzyl ammonium cations include cetyl dimethyl benzyl ammonium, lauryl dimethyl benzyl ammonium, myristyl dimethyl benzyl ammonium, stearyl dimethyl benzyl ammonium and arachidyl dimethyl benzyl ammonium.

In other embodiments, the aliphatic benzyl alkyl ammonium cation may comprise, but is not limited to, alkyl ethyl methyl benzyl ammonium cations. Specific non-limiting examples of alkyl ethyl methyl benzyl ammonium cations include cetyl ethyl methyl benzyl ammonium, lauryl ethyl methyl benzyl ammonium, myristyl ethyl methyl benzyl ammonium, stearyl ethyl methyl benzyl ammonium and arachidyl ethyl methyl benzyl ammonium.

### Dialiphatic Dialkyl Ammonium

Further examples of QACs that may be used for the ionic liquids are dialiphatic dialkyl ammonium cations. A dialiphatic dialkyl ammonium cation is a compound comprising two aliphatic groups and two alkyl groups which are bound to the nitrogen atom. The aliphatic groups may be the same or different and may comprise any aliphatic group as described above. The alkyl groups may be the same or different and may comprise any alkyl group as described above. In the disclosed dialiphatic dialkyl ammonium cations, the two aliphatic groups may contain ten or more carbon atoms and the two alkyl groups may contain less than ten carbon atoms. Alternatively, the two aliphatic groups may contain less than ten carbon atoms and the two alkyl groups may contain ten or more carbon atoms. One or more types of dialiphatic dialkyl ammonium cations may be used in the ionic liquids.

In specific embodiments, the dialiphatic dialkyl ammonium cation may be didodecyldimethyl ammonium, ditetradecyldimethyl ammonium, dihexadecyl-dimethyl ammonium and the like, including combinations thereof.

### Other Cations

In a specific embodiment of the present invention, choline esters (e.g. acetylcholine) may be used as counterions. Choline esters may be prepared by esterification of a compound having a carboxyl group or by transesterification of a compound having an ester group with a choline moiety. Further examples of choline esters are bethanechol, carbachol, citicolin, methacholine, succinylmonocholine, suxamethonium chloride. In a specific embodiment, choline may be used as a counterion.

Further examples of preferred cations are (2-hydroxyethyl)-dimethylundecyloxymethyl ammonium, (2-acetoxyethyl)-heptyloxymethyldimethyl ammonium, (2-acetoxyethyl)-dodecyloxymethyldimethyl ammonium and mepenzolate.

In a further embodiment, the cationic counterions are derived from an arylamine, which preferably is ethyleneamine or piperazine.

In a further embodiment, the cationic counterions are derived from an alkylamine containing hydroxyl groups, which preferably is diethanolamine, triethanolamine, tromethamine or N-methylglucamine.

In a preferred embodiment, ionic liquids based on highly lipophilic amine bases, such as benzathine, 4-phenyl cyclohexylamine, benethamine and hydrabamine, are used.

In a further preferred embodiment, the ionic liquid comprises a rotigotine anion and an N,N,N-trimethylethanol ammonium cation (choline).

### Anions as Counterions

In other embodiments of this aspect of the present invention, an organic acid is used for the ionic liquid. Preferably, said organic acid is of the formula R—COOH, wherein R represents a saturated or monounsaturated, branched or unbranched C₃-C₁₆ hydrocarbon residue. Said hydrocarbon residue has 3 to 16, more preferably 5 to 16, carbon atoms. In a further embodiment, R preferably represents a saturated, monounsaturated or polyunsaturated, unbranched C₃-C₁₆ hydrocarbon residue, more preferably a C₅-C₁₆ hydrocarbon residue.

Examples for preferred acids of the formula R—COOH are propionic acid, butyric acid, pentanoic acid, hexanoic acid, heptanoic acid, octanoic acid, nonanoic acid, decanoic acid, undecanoic acid, lauric acid, tridecanoic acid, myristic acid, pentadecanoic acid, and palmitic acid. Octanoic acid is particularly preferred.

In a further embodiment, the organic acid is of the formula R′—COOH, wherein R′ represents a polyunsaturated, branched or unbranched C₁₈-C₂₂ hydrocarbon residue.

Examples for preferred acids of the formula R′—COOH are linoleic acid, alpha linoleic acid, arachidonic acid, eicosapentaenoic acid and docosahexaenoic acid. Linoleic acid is particularly preferred. Further suitable anions are obtainable from long-chain alkyl sulfonic acids (e.g. lauryl sulfonic acid), saccharin or acesulfame.

In particular embodiments of this aspect of the present invention, the anionic counterion is derived from a lipophilic monovalent acid. Preferably, the anionic counterion of a lipophilic monovalent acid represents at least 50% of the anions in the ionic liquid. The lipophilic monovalent acid preferably is octanoic acid.

### Methods for Preparing Ionic Liquids

Basically, there are three methods for preparing an ionic liquid:
(1) Metathesis of a salt of the desired cation (e.g. a halogenide salt) with a salt of the desired anion (e.g. a transition metal (e.g. silver) salt, salts of the groups I or II, or an ammonium salt). Such reactions may be carried out with many types of salts.
(2) An acid/base neutralization reaction.
(3) Exchange of the relevant ion by the addition of a stronger acid or base.

The combination of rotigotine with a counter cation in a solvent with optional heating may thus serve for the preparation of ionic liquids.

### Preparation of an Ionic Liquid of Rotigotine Using an Organic Acid:

The ionic liquid of rotigotine and an organic acid as the anion-forming reagent may be prepared by mixing rotigotine and acid to obtain the ionic liquid. In one embodiment, the mixing step is carried out in the absence of any solvent. This is advantageous because a step of removing the solvent after the formation of the ionic liquid may thus be omitted and the method is furthermore ecologically friendly. It is a further advantage of the absence of any solvent that the ionic liquid thus obtained does not contain any residual solvent.

Alternatively, rotigotine and the organic acid may also be mixed in the presence of one or more solvents. In this case, either rotigotine alone or rotigotine together with the acid may be dissolved in the same solvent or in different solvents before the mixing step is carried out. For instance, rotigotine may be dissolved in a solvent, followed by the addition of the acid to the solution thus obtained. Suitable solvents are, e.g., water, alcohols, ethers, ketones, chlorinated solvents, and mixtures thereof. Preferred solvents are ethers and ketones.

### Preparation of an Ionic Liquid of Rotigotine Using an Amine:

The ionic liquid of rotigotine and an amine as the cation-forming reagent may be prepared by mixing rotigotine or a suitable acid addition salt of rotigotine and the corresponding amine and optionally an additional base in the presence of one or more solvents. In this case, rotigotine, the amine and/or the additional base may be dissolved in the same solvent or in different solvents before the mixing step is carried out. For instance, rotigotine may be dissolved in a solvent, followed by the addition of the amine and the optional additional base to the solution thus obtained. Suitable solvents are e.g. water, alcohols, ethers, ketones, chlorinated solvents, and mixtures thereof. Preferred solvents are alcohols. The optional additional base is a strong base, such as e.g. hydroxides or hydrides. Preferred optional additional bases are sodium hydroxide and potassium hydroxide.

### EXAMPLE 1

### Ionic Liquid Containing a Rotigotine Cation and an Octanoate Anion

### EXAMPLE 1a

To a solution of rotigotine (1 equivalent) in diisopropyl ether (5-fold volume) 1 equivalent of octanoic acid is added under nitrogen. After stirring (1 h) the solvent is removed in a vacuum at 30° C. By high-vacuum drying for 2 h a quantitative yield of oil is obtained.

### EXAMPLE 1b

Rotigotine (1 equivalent) is added to one equivalent of octanoic acid in a glass vial. The mixture is shaken for 1 h until a homogeneous oil is formed.

### EXAMPLE 2

### Ionic Liquid Containing a Rotigotine Anion and a Choline Cation

### EXAMPLE 2a

Rotigotine (1 equivalent) is dissolved in methanol (5 volumes) in a round bottom flask. Potassium hydroxide (1 equivalent) and choline chloride (1 equivalent) are added and the mixture is stirred for 1 h at room temperature. Acetone (5 volumes) is added and the solution is filtered through a fine filter. The filtrate is concentrated in a vacuum at room temperature and subsequently high-vacuum dried. The product thus obtained is a quantitative yield of an oily residue.

### EXAMPLE 2b

Rotigotine hydrochloride (1 equivalent) is dissolved in methanol (5 volumes) in a round bottom flask. Potassium hydroxide (2 equivalents) and choline chloride (1 equivalent) are added and the mixture is stirred for 1 h at room temperature. Acetone (5 volumes) is added and the solution is filtered through a fine filter. The filtrate is concentrated in a vacuum at room temperature and subsequently high-vacuum dried. The product thus obtained is a quantitative yield of an oily residue.

1. A transversal therapeutic system (TTS) containing an active ingredient rotigotine or a pharmaceutically acceptable salt thereof and having a backing layer (1) which is impermeable to the active ingredient, a matrix layer (2) containing the active ingredient and a removable protective sheet (4) which is intended for removal before the patch is applied to the skin, characterized in that a first interlayer (3) is disposed with areal coverage between the matrix layer (2) and the removable protective sheet (4).

2. The TTS according to claim 1, wherein the first interlayer (3) consists of a pressure-sensitive polymer adhesive.

3. The TTS according to claim 1, wherein the matrix layer (2) consists of non-adhesive polymers having long aliphatic chains and has a second interlayer (5) consisting of the same material as the first interlayer (3).

4. The TTS according to claim 1, wherein the thickness of the interlayer(s) is between 4 μm and 25 μm.

5. The TTS according to claim 1, wherein the active ingredient is rotigotine in the free base form.

6. The TTS according to claim 1, wherein the active ingredient is rotigotine in the form of an ionic liquid, wherein the ionic liquid comprises:
(a) at least one rotigotine cation and at least one type of counterion obtained from an organic compound; or
(b) at least one rotigotine anion and at least one type of counterion obtained from an organic compound.

7. The TTS according to claim 6, wherein the ionic liquid comprises at least one rotigotine cation and at least one type of counterion, wherein the counterion is an anion of an organic acid.

8. The TTS according to claim 7, wherein the organic acid is selected from:
a compound of the formula R—COOH, wherein R represents a saturated or monounsaturated, branched or unbranched C₃-C₁₆ hydrocarbon residue; or
(ii) a compound of the formula R′—COOH, wherein R′ represents a polyunsaturated, branched or unbranched C₁₈-C₂₂ hydrocarbon residue.

9. The TTS according to claim 7, wherein the organic acid is selected from the group consisting of propionic acid, butyric acid, pentanoic acid, hexanoic acid, heptanoic acid, octanoic acid, nonanoic acid, decanoic acid, undecanoic acid, lauric acid, tridecanoic acid, myristic acid, pentadecanoic acid, palmitic acid, and unsaturated analogs thereof.

10. The TTS according to claim 7, wherein the organic acid is octanoic acid.

11. The TTS according to claim 6, wherein the ionic liquid comprises at least one rotigotine anion and at least one type of counterion, wherein the counterion is an amine-derived cation.

12. The TTS according to claim 11, wherein said amine-derived cation is selected from among:
(i) a quaternary amine of the formula (I)
R¹R²R³R⁴N⁺ (I)
wherein R¹, R², R³, and R⁴ each independently may represent H, an optionally substituted C₁-C₅ alkyl or alkenyl, an optionally substituted C₆-C₁₀ cycloalkyl, an optionally substituted C₆-C₁₂ aryl, or an optionally substituted C₇-C₁₂ aralkyl, or, alternatively, wherein R¹ and R² together represent an optionally substituted C₄-C₁₀ alkylene group and form a 5- to 11-membered heterocyclic ring with the nitrogen atom of formula (I), further wherein the term “optionally substituted” indicates that the respective group is either substituted with one or more residues selected from the group consisting of OH, SH, SR⁵, Cl, Br, F, I, NH₂, CN, NO₂, COOR⁵, CHO, COR⁵ and OR⁵, wherein R⁵ represents a C₁-C₁₀ alkyl or cycloalkyl group, or is not substituted;
(ii) a tetraalkyl ammonium ion;
(iii) an aliphatic heteroaryl ammonium ion;
(iv) a dialiphatic dialkyl ammonium cation.

13. The TTS according to claim 12, wherein said amine-derived cation comprises a quaternary amine of the formula (I), further wherein R¹ is a C₁-C₅ alkyl or alkenyl.

14. The TTS according to claim 11, wherein the amine of the amine-derived cation is selected from the group consisting of diethanolamine triethanolamine, ethyleneamine, trimethamine, N-methylglucamine, piperazine, benzathine, 4-phenyl cyclohexylamine, benethamine and hydrabamine.

15. The TTS according to claim 11, wherein the amine-derived cation comprises an N,N,N-trimethylethanol ammonium cation.

16. The TTS according to claim 1, comprising a rotigotine salt, wherein at least 1% by weight of the salt represents an ionic liquid comprising:
(a) at least one rotigotine cation and at least one type of counterion obtained from an organic compound; or
(b) at least one rotigotine anion and at least one type of counterion obtained from an organic compound.

17. The TTS according to claim 1, wherein the active ingredient is present in the matrix layer (2) in a completely dissolved state.

18. The TTS according to claim 1, wherein the solubility of the interlayer with respect to the active ingredient is between 4 and 30% by weight.

19. The TTS according to claim 1, wherein the content of the active ingredient rotigotine in the matrix layer (2) is between 4 and 30% by weigh as related to the free base.

20. The TTS according to claim 1, wherein the solubility of the interlayer(s) with respect to the active ingredient is between 5% and 15% of the solubility of the matrix layer with respect to the active ingredient.

21. The TTS according to claim 1, wherein the ratio of the thickness of the matrix layer (2) and the thickness of the first interlayer (3) is between 20:1 and 3:1.

22. The TTS according to claim 1, wherein the TTS has a size of between 5 cm² and 100 cm².

23. The TTS according to claim 1, wherein the TTS has an adhesive strength of between 5 N/25 mm² and 100 N/25 mm².

24. The TTS according to claim 1, wherein the TTS is suitable for an administration of rotigotine over 12 h to 3 days.

25. The TTS according to claim 1, wherein the release rate is in a range between 0.05 μg/h and 1 μg/h over a substantial portion of the administration period.

26. The TTS according to claim 1, wherein the TTS provides a mean maximum plasma concentration (Cₘₐₓ) of rotigotine between 0.1 ng/mL and 150 ng/mL over a substantial portion of the administration period.

27. The TTS according to claim 3, wherein the long aliphatic chains of the non-adhesive polymers of the matrix layer comprise polyacrylate esters with 4 to 8 carbon atoms.

28. The TTS according to claim 4, wherein the thickness of the interlayer(s) is between 8 μm and 20 μm.

29. The TTS according to claim 4, wherein the thickness of the interlayer(s) is between 10 μm and 15 μm.

30. The TTS according to claim 12, wherein said amine-derived cation is quaternary amine of the formula (I) substituted with 1 to 6 residues selected from OH, SH, SR⁵, Cl, Br, F, I, NH₂, CN, NO₂, COOR⁵, CHO, COR⁵ and OR⁵.

31. The TTS according to claim 16, comprising a rotigotine salt, wherein at least 2% by weight of the salt represents said ionic liquid.

32. The TTS according to claim 16, comprising a rotigotine salt, wherein at least 5-20% by weight of the salt represents said ionic liquid.

33. The TTS according to claim 16, comprising a rotigotine salt, wherein at least 30-50% by weight of the salt represents said ionic liquid.

34. The TTS according to claim 18, wherein the solubility of the interlayer with respect to the active ingredient is between 6 and 20% by weight.

35. The TTS according to claim 18, wherein the solubility of the interlayer with respect to the active ingredient is between 8 and 15% by weight.

36. The TTS according to claim 19, wherein the content of the active ingredient rotigotine in the matrix layer (2) is between 6 and 20% by weight, even more preferably between 8 and 15% by weight as related to the free base.

37. The TTS according to claim 19, wherein the content of the active ingredient rotigotine in the matrix layer (2) is between 8 and 15% by weight as related to the free base.

38. The TTS according to claim 20, wherein the solubility of the interlayer(s) with respect to the active ingredient is between 5% and 10% of the solubility of the matrix layer with respect to the active ingredient.

39. The TTS according claim 21, wherein the ratio of the thickness of the matrix layer (2) and the thickness of the first interlayer (3) is between 10:1 and 5:1.

40. The TTS according to claim 22, wherein the TTS has a size of between 10 cm² and 50 cm².

41. The TTS according to claim 23, wherein the TTS has an adhesive strength of between 10 N/25 mm² and 50 N/25 mm².

42. The TTS according to claim 24, wherein the TTS is suitable for an administration of rotigotine over 24 h to 48 h.

43. The TTS according to claim 25, wherein the release rate is in a range between 0.1 μg/h and 0.5 μg/h over a substantial portion of the administration period.

44. The TTS according to claim 26, wherein the TTS provides a mean maximum plasma concentration (Cₘₐₓ) of rotigotine between 0.5 ng/mL and 100 ng/mL over a substantial portion of the administration period.
